(19) United States
(12) Patent Application Publication
(10) Pub. No.: US 2020/0410665 A1
(43) Pub. Date: Dec. 31, 2020

(54) CONTROL OF AN IMAGE-CAPTURING DEVICE ALLOWING THE COMPARISON OF TWO VIDEOS

(71) Applicant: Emmanuel ELARD, Saint Mande (FR)

(72) Inventor: Emmanuel ELARD, Saint Mande (FR)

(21) Appl. No.: 16/082,098

(22) PCT Filed: Mar. 1, 2017
(86) PCT No.: PCT/FR2017/050456

§ 371 Date: Sep. 9, 2020

(30) Foreign Application Priority Data
Mar. 1, 2016 (FR) 1651695

(51) Int. Cl. G06T 7/00; A61B 5/107; G03B 15/14; H04N 5/232

(52) U.S. Cl. CPC G06T 7/0012; A61B 5/1079; H04N 5/23222; G06T 7/97; G03B 15/14

(57) ABSTRACT

Some embodiments are directed to a method of video image-capturing by an image-capturing apparatus secured to a support, the support being designed to be set into rotation about a zone intended to accommodate a patient by an electric motor controlled by a control unit.

### CROSS REFERENCE TO RELATED APPLICATIONS

This application is a national phase filing under 35 C.F.R. § 371 of and claims priority to PCT Patent Application No. PCT/FR2017/050456, filed on Mar. 1, 2017, which claims the priority benefit under 35 U.S.C. § 119 of French Patent Application No. 1651695, filed on Mar. 1, 2016, the contents of each of which are hereby incorporated in their entireties by reference.

### BACKGROUND

Some embodiments of the presently disclosed subject matter relate to the field of video image-capturing in the context of medicine and cosmetic surgery, orthodontics, beauty or fitness services, and more generally fields requiring an accurate comparison of images before/after a procedure.

Some other embodiments relate more particularly to an electronic device and software for such video image captures.

In the field of cosmetic medicine and surgery, practitioners conventionally capture images of the face, or of a part of the body of patients, before and after a procedure.

Standard in respect of image capture conditions before/after a procedure have been put in place to ensure relevant observations, as reiterated in the publication “Photographic Standards in Plastic Surgery”, 2006, Plastic Surgery Education Foundation, USA. Image captures before/after a procedure must or should be obtained using the same equipment and the same procedures. The image-capturing apparatus, lighting, magnification, framing, position of the patient and the preparation thereof must or should be consistent.

The equipment used is most frequently similar to that of a professional photographer in his/her photography studio, as demonstrated by the chapter “Photography in Facial Plastic Surgery” in the treatise “Advanced Therapy in Facial Plastic & Reconstructive Surgery”, J. Regan Thomas, p. 31, 2010, PMPH, USA. This equipment comprises a chair intended for the patient placed in front of a uniform background, an image-capturing device on a tripod type mount, lights and reflectors.

Most practitioners create static image captures, under various standardised angles. However, panoramic, or even stereoscopic, images, or before/after videos, would greatly enhance the perception that the patient could have of the benefit of his/her procedure. It is extremely difficult to create video sequences before/after a procedure manually such that the before/after video sequences may be superimposed with a view to the image-by-image comparison thereof. Therefore, there emerges a need for practitioners to equip themselves with automated video image-capturing systems.

In the related art, automated systems suitable for capturing images of this type are known, for example, the stereoscopic remote viewing system described in the European patent application EP0218751. In this system, a video camera mounted on a carriage, which moves along a semi-circular rail, makes it possible to obtain video sequences of an object placed at the centre of the rail. However, this system intended for industry is complex and costly and is not suitable for use in the field of cosmetic medicine and surgery.

### SUMMARY

Some embodiments of the presently disclosed subject matter therefore provide a solution for remedying at least partially the drawbacks mentioned above.

One objective of some embodiments of the presently disclosed subject matter is that of helping simplify video image-capturing systems intended for cosmetic medicine and surgery, particularly by reducing the number of components, which makes it possible to reduce the production costs of such systems and increase the robustness thereof. For example, the solution proposed by some embodiments of the presently disclosed subject matter economise on connectors and sensors.

Some embodiments of the presently disclosed subject matter therefore relate to a method of video image-capturing by an image-capturing apparatus secured to a support, the support being designed to be set into rotation about a zone intended to accommodate a patient by an electric motor slaved by a control unit, the method comprising steps of
transmission to the control unit of an order to position the support in a first position, and display of a first photographic image captured by the apparatus, on a man-machine interface;
after reception of a command via the man-machine interface, transmission to the control unit of an order to position the support in a second position; and display of a second photographic image captured by the apparatus, on the main-machine interface
after reception of a command via the man-machine interface, transmission to the control unit of an order to trigger the rotation of the support and triggering of the recording of a first video;
transmission to the control unit of an order to position the support in the first position, and display of a first new photographic image as well as, in transparency, an image extracted from the first video corresponding to the first position, on a man-machine interface;
after reception of a command via the man-machine interface, transmission to the control unit of an order to position the support in the second position; and display of a second new photographic image as well as, in transparency, an image extracted from the first video corresponding to the second position, on the man-machine interface;
after reception of a command via the man-machine interface, transmission to the control unit of an order to trigger the rotation of the support and triggering of the recording of a second video;
display of the first and second video on the man-machine interface.

According to some embodiments, the presently disclose subject matter comprise one or a plurality of the following features which may be used separately or in partial combination or in complete combination with one another:
the first and second positions are separated by an angle of at least 90 degrees;
the first and second videos are displayed in two windows of the same screen, opposite one another and synchronised in respect of time;
the first and second videos are displayed in a single window and separated by a separator the position whereof may be modified dynamically by the user;
the rotation of the support includes at least one stop.

The presently disclosed subject matter further relate to a video image-capturing device (1) comprising an image-capturing apparatus (2) secured to a support (3), the support being designed to be set into rotation about a zone intended to accommodate a patient by an electric motor slaved by a control unit and comprising software means provided for
transmitting to the control unit an order to position the support in a first position, and displaying a first photographic image captured by the apparatus, on a man-machine interface;
after reception of a command via the man-machine interface, transmitting to the control unit an order to position the support in a second position; and displaying a second photographic image captured by the apparatus, on the main-machine interface;
after reception of a command via the man-machine interface, transmitting to the control unit an order to trigger the rotation of the support and trigger the recording of a first video;
transmitting to the control unit an order to position the support in the first position, and displaying a first new photographic image as well as, in transparency, an image extracted from the first video corresponding to the first position, on a man-machine interface;
after reception of a command via the man-machine interface, transmitting to the control unit an order to position the support in a second position; and displaying a second new photographic image as well as, in transparency, an image extracted from the first video corresponding to the second position, on the man-machine interface;
after reception of a command via the man-machine interface, transmitting to the control unit an order to trigger the rotation of the support and trigger the recording of a second video;
displaying the first and second video on the man-machine interface.

According to some embodiments, the presently disclosed subject matter comprise one or a plurality of the following features which may be used separately or in partial combination or in complete combination with one another:
the software means are envisaged to transmit orders to the control unit such that the first and second positions are separated by an angle of at least 90 degrees;
the man-machine interface belongs to an electronic apparatus having a radio interface enabling communication with the control unit;
the electronic apparatus is the image-capturing apparatus, the image-capturing apparatus further having a touch screen;

Further features and advantages of the presently disclosed subject matter will emerge on reading the following description of some embodiments of the presently disclosed subject matter, given by way of example and with reference to the appended figures.

### BRIEF DESCRIPTION OF THE FIGURES

FIG. 1 illustrates an embodiment of an image-capturing system suitable for implementing the device according to the presently disclosed subject matter.

FIG. 2 illustrates schematically an architecture which may be set up to implement the principles of some embodiments of the presently disclosed subject matter.

FIG. 3 illustrates the method according to some embodiments of the presently disclosed subject matter very schematically.

FIGS. 4a and 4b illustrate schematically two embodiments of display of the first and second videos.

### DETAILED DESCRIPTION OF EXEMPLARY EMBODIMENTS

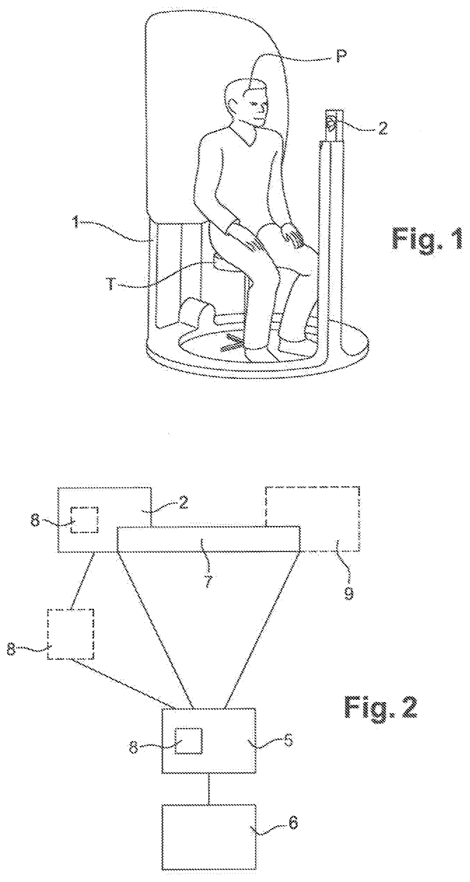

FIG. 1 illustrates an embodiment of an image-capturing system suitable for implementing the device 1 according to the presently disclosed subject matter.

A patient P is set up in the seated position on a stool T, within a zone intended to accommodate the patient.

The device comprises a fixed base spread on the horizontal plane, and a circular rail secured to the base and also horizontal. On this rail, may move a horizontal platform 4 actuated by an electric motor and comprising a support 3.

An electric motor is suitable for setting into rotation the platform, and consequently the support 3 about the central zone containing the stool T.

An image-capturing apparatus 2 is positioned on the support 3. Various attachment means may be envisaged so that the apparatus 2 is held securely during the rotation of the support 3 about the patient P. It must or should be positioned in a secured manner, such that the position of the lens accurately follows the rotation of the support 3, and the lens is continuously oriented towards the patient P.

As such, the image-capturing apparatus may rotate about the patient according to an angle which may be variable according to the embodiments and configurable. Preferentially, this sweep may cover an angle at least equal to 180° so as to enable for example one front photographic image and two profile photographic images of the patient P. It may cover 360° so as to enable the full capture of the patient P.

The image-capturing apparatus 2 may be a “smartphone” type mobile communication terminal, or indeed a video camera, etc. It may have a man-machine interface suitable for, on one hand, displaying the videos, and, on the other, receiving commands from a user.

FIG. 2 illustrates schematically an architecture which may be set up to implement the principles of some embodiments of presently disclosed subject matter

The apparatus 2 is functionally connected to a control unit 5 intended to slave the motor 6 suitable for setting into rotation the support 3.

A man-machine interface 7 is also provided to enable at least, on one hand, the display of recorded videos and, on the other, reception of the commands from the user of the device 2.

This man-machine interface may be totally or partially implemented by the image-capturing apparatus 2, for example in the case whereby the latter is a “smartphone” type communication terminal or a digital tablet, etc.

The man-machine interface 7 may also be partially (or completely) implemented by one or a plurality of further apparatuses 9. It may include a control and/or display panel physically linked to the device 1. It may also include an apparatus not physically but functionally linked for example by means of a radio link, such as a remote control.

The man-machine interface may comprise a touch screen, particularly in the case of the interface part implemented by the video image-capturing apparatus 2.

The commands inherent to the presently disclosed subject matter will be seen subsequently, but it may be mentioned for the clarity of the disclosure at this stage that the man-machine interface may further be suitable for starting up or shutting down the device 1, setting the speed of the motor 6, the direction of rotation thereof, the angular range of rotation, a number of stops during video acquisition, the position and duration of the stops, etc.

The video image-capturing device also comprises software means 8. These software means may be implemented partially or completely in the image-capturing apparatus 2, particularly when the latter is of the “smartphone” or digital tablet type. They may also be implemented in a processing device contained in the device 1, which may be co-located with the control unit 5, or indeed offset on a separate processing platform to that implementing the control unit 5.

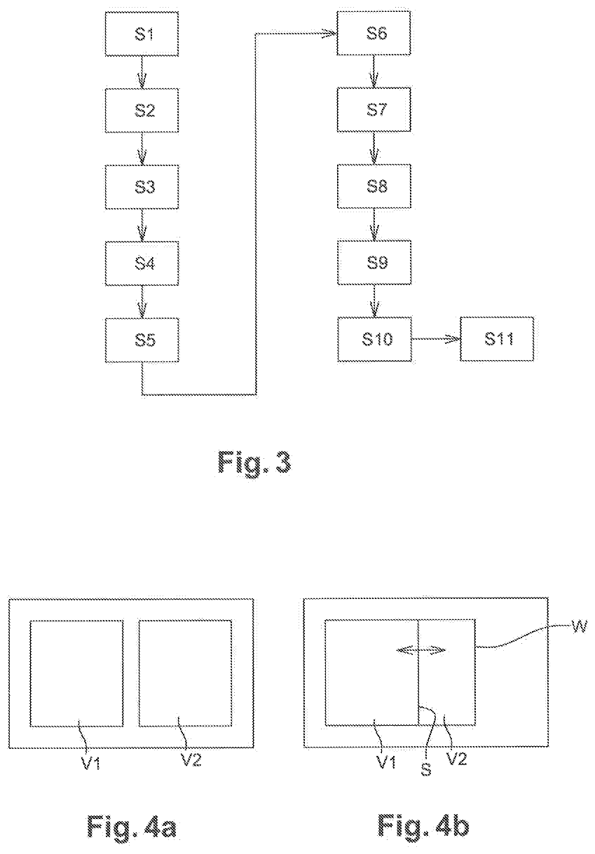

FIG. 3 illustrates the method according to some embodiments of the presently disclosed subject matter very schematically.

A first preliminary step, not shown in the figure, includes carrying out a white balance, so as to calibrate the image-capturing apparatus 2. For this purpose, a test chart or a mere blank sheet may be presented in front of the apparatus 2.

Then, in a first step S1, the software means transmit to the control unit 5 an order to position the support 3 in a first position, and trigger the display of a first photographic image captured by the apparatus, on the man-machine interface 7.

This first position may for example correspond to a front view of the patient P. The support 3 is therefore positioned facing the patient.

The precise positioning may be guided by a screen associated with the image-capturing apparatus 2 whereon may be displayed crosshairs, for example in the form of a cross including a vertical line intended to be positioned on the bridge of the nose of the patient P, and a horizontal line intended to be positioned on an imaginary line passing through the centre of the eyes of the patient P.

An additional screen may be provided to be oriented towards the patient to enable the patient to position him/herself more easily.

Once the patient is positioned, the user may trigger a command via the man-machine interface 7. This command may be triggered by merely tapping on the screen of the apparatus 2, but further control means may obviously be deployed (voice control, button on screen or on a control panel, etc.)

The step S2 indicates the reception of this command by the software means which may then trigger, in a step S2, the transmission to the control unit of an order to position the support 3 in a second position, and trigger the display of a second photographic image captured by the apparatus, on the man-machine interface 7.

This second position is separate from the first position. The angle between the first and second positions must or should be relatively large, so as to enable the recording of significant videos at the subsequent steps, i.e. showing as completely as possible the patient's face from varied angles enabling an assessment or a diagnosis. For example, the separation may be greater than or equal to 90° so as to be able to obtain a front photographic image and a profile photographic image of the patient P, the second position then corresponding to this profile view. The angular separation may be greater, for example 180° so as to capture a front view and a view of each profile of the patient. It may go up to 360°.

The second photographic image may be displayed in the same way as the first image in the step S1. In particular, the same crosshairs may be set up for the precise positioning of the patient P with respect to the lens of the image-capturing apparatus 2. A profile view makes it possible to adjust the inclination of the patient's head, so that it is not tilted too much either upwards or downwards.

Once the patient has been positioned, a command may be transmitted via the man-machine interface and received, in a step S4, by the software means.

This command may be similar to the preceding command; it may particularly include merely tapping on the touch screen of the image-capturing apparatus 2.

The reception of this command by the software means triggers the start of a second phase including recording a first video.

In a step referenced S5 in FIG. 3, the software means transmit to the control unit 5 an order to trigger rotation of the support 8 between the second and the first positions.

This rotation may be continuous or interrupted by pauses. These pauses may enable the patient to smile or make other facial muscle movements helping enhance the semantic content of the video. Indeed, it may be of interest to have images, from different angles, of the face smiling or making certain expressions, to increase the volume of information and enable better decision-making and diagnosis by the user.

Instructions in this respect may be provided to the patient either by the user, or by the device 1. In the latter case, the man-machine interface 7 may provide instructions verbally, using voice synthesis means in particular, or visually by a screen oriented towards the patient.

The video enables dynamic image capture of the patient P, and therefore comprises much richer semantic content than a mere set of static image captures. Indeed, for a relevant diagnosis, it may be necessary to have not static images, but video images showing the transitions from one expression to another, or indeed a continuity of viewing angles.

The rotation may be in a single direction, for example from the second to the first position, or include an outward and return travel and any other travel between the first and second positions.

At the end of the travel, the support 3 may be stopped by the control means 5 and the recording may be interrupted. A first video is thereby obtained.

The treatment of the patient may then take place. The first video therefore includes a video of before the treatment. This first video is stored in a memory associated with the device 1.

A third phase commences after the treatment in order to record a second video, representing an after-treatment video.

This third phase commences with the reception of a relevant command by the man-machine interface 7.

The software means may then, in a step S6, transmit to the control unit 5 an order to position the support 3 in the first position. They then trigger the display on the man-machine interface 7 of a new photographic image captured by the apparatus 2 as well as, in transparency, an image extracted from the first video and corresponding to the same first position.

As such, it is possible to reposition the patient P in the same way as during the first phase, but instead of doing so based on crosshairs, the positioning is guided by an extraction of the first video. It is thereby possible to ensure correspondence of the facial contours of the patient P and/or of certain characteristic traits (eyes, bridge of nose, etc.), such that it can be ensured that the second video captured will enable a precise and relevant comparison with the first video.

A further white balance setting may be carried out, but it may be proposed to use the calibration determined during the first phase. If the environmental conditions remain constant, this makes it possible to obtain identical colorimetric characteristics on both videos obtained and therefore facilitate comparison.

When the precise positioning is carried out, in the same way as previously, a command may be transmitted via the man-machine interface and received, in a step S7, by the software means. This command may be, as described above, of different types and particularly merely a tap on the touch screen of the apparatus 2.

In a step S8, and following this command, the software means transmit to the control unit 5 an order to position the support 3 in the second position.

They then trigger the display on the man-machine interface 7 of a second new photographic image captured by the apparatus 2 as well as, in transparency, an image extracted from the first video and corresponding to the same second position.

As such, it is possible to reposition the patient P as previously by means of an extraction of the first video. It is thereby possible to ensure correspondence of the facial contours of the patient P and/or of certain characteristic traits (eyes, bridge of nose, etc.) in the same way as for the first position.

The first and second positions are the same, precisely, as those created in the first phase, such that the videos before and after treatment correspond to the same travel of the apparatus 2 and the videos are thereby completely comparable.

Once the patient has been positioned, a command may be transmitted via the man-machine interface and received, in a step S9, by the software means. This command may be similar to the preceding command; it may particularly include merely tapping on the touch screen of the image-capturing apparatus 2.

In the same way as previously, a fourth phase may then be triggered by the reception of this command by the software means.

In a step reference S10 in FIG. 3, the software means transmit to the control unit 5 an order to trigger a rotation of the support 8 between the second and the first positions. This rotation preferentially follows the same travel as for the recording of the first video, such that the first and second videos are comparable.

In a step S11, both videos are displayed on the man-machine interface. This step may be concomitant with the step S10, i.e. the second video is displayed at the same time as the recording thereof, in real time or in slightly delayed time. It may take place alternatively following same, and insofar as both videos may be stored in a memory of the device 1, they may be displayed a number of times.

FIGS. 4a and 4b illustrate two embodiments of display of the first and second videos.

In the embodiment in FIG. 4a, two zones of a screen (for example two windows) each have one of the two videos v1, v2. The videos are synchronised such that, at each time, images corresponding to identical positions of the support 3 are shown opposite one another enabling easy comparison for the user.

Control means (play, stop, rewind, slow motion, etc.) are used to control both videos simultaneously.

In the embodiment in FIG. 4b, the same zone w, or window, can be used to view both videos v1, v2, simultaneously. A separator S separates the display of both videos. The position thereof may be dynamically modified by the user, according to the arrows shown, in order to show complementary regions of each of the two videos. For example, if the separator is dragged to one end, only one of the two videos will be displayed in the window w.

Obviously, the presently disclosed subject matter is not limited to the examples and to the embodiment described and represented, but it is suitable for numerous alternative embodiments accessible to those of ordinary skill in the art.

1. A method of video image-capturing by an image-capturing apparatus secured to a support, the support being designed to be set into rotation about a zone intended to accommodate a patient by an electric motor slaved by a control unit, the method comprising steps of:
transmission to the control unit of an order to position the support in a first position, and display of a first photographic image captured by the apparatus, on a man-machine interface;
after reception of a command via the man-machine interface, transmission to the control unit of an order to position the support in a second position; and display of a second photographic image captured by the apparatus, on the main-machine interface
after reception of a command via the man-machine interface, transmission to the control unit of an order to trigger the rotation of the support and triggering of the recording of a first video;
transmission to the control unit of an order to position the support in the first position, and display of a first new photographic image as well as, in transparency, an image extracted from the first video corresponding to the first position, on a man-machine interface;
after reception of a command via the man-machine interface, transmission to the control unit of an order to position the support in the second position; and display of a second new photographic image as well as, in transparency, an image extracted from the first video corresponding to the second position, on the man-machine interface;
after reception of a command via the man-machine interface, transmission to the control unit of an order to trigger the rotation of the support and triggering of the recording of a second video; and
display of the first and second video on the man-machine interface.

2. The method according to claim 1, wherein the first and second positions are separated by an angle of at least 90 degrees.

3. The method according to claim 1, wherein the first and second videos are displayed in two windows of the same screen, opposite one another and synchronised in respect of time.

4. The method according to claim 1, wherein the first and second videos are displayed in a single window and separated by a separator the position whereof may be modified dynamically by the user.

5. The method according to claim 1, wherein the rotation of the support includes at least one stop.

6. A video image-capturing device comprising an image-capturing apparatus secured to a support, the support being designed to be set into rotation about a zone intended to accommodate a patient by an electric motor slaved by a control unit and comprising software means provided for
transmitting to the control unit an order to position the support in a first position, and displaying a first photographic image captured by the apparatus, on a man-machine interface;
after reception of a command via the man-machine interface, transmitting to the control unit an order to position the support in a second position; and displaying a second photographic image captured by the apparatus, on the main-machine interface
after reception of a command via the man-machine interface, transmitting to the control unit an order to trigger the rotation of the support and trigger the recording of a first video;
transmitting to the control unit an order to position the support in the first position, and displaying a first new photographic image as well as, in transparency, an image extracted from the first video corresponding to the first position, on a man-machine interface;
after reception of a command via the man-machine interface, transmitting to the control unit an order to position the support in the second position; and displaying a second new photographic image as well as, in transparency, an image extracted from the first video corresponding to the second position, on the man-machine interface;
after reception of a command via the man-machine interface, transmitting to the control unit an order to trigger the rotation of the support and trigger the recording of a second video;
displaying the first and second video on the man-machine interface.

7. The device according to claim 6, wherein the software means are envisaged to transmit orders to the control unit such that the first and second positions are separated by an angle of at least 90 degrees.

8. The device according to claim 6, wherein the man-machine interface belongs to an electronic apparatus having a radio interface enabling communication with the control unit.

9. The device according to claim 8, wherein the electronic apparatus is the image-capturing apparatus, the image-capturing apparatus further having a touch screen.
